(19) United States
(12) Patent Application Publication
(10) Pub. No.: US 2025/0359855 A1
(43) Pub. Date: Nov. 27, 2025

(54) A DEVICE AND METHOD FOR SINONASAL SECRETION COLLECTION

(71) Applicant: Diag-Nose Medical Pty Ltd, Port Melbourne (AU)

(72) Inventors: Shaikh Salah El Din Rostom, Hawthorn East (AU); Brian Shih-Ning Wang, Houston, TX (US); David Melicio Yen, Bethlehem, PA (US); Jonathan Limpah, Parkwood (AU); Adam Michael Damry, Braddon (AU)

(73) Assignee: Diag-Nose Medical Pty Ltd, Port Melbourne (AU)

(21) Appl. No.: 18/874,667

(22) PCT Filed: Jun. 14, 2023
(86) PCT No.: PCT/AU2023/050525

§ 371 Date: Dec. 13, 2024

Related U.S. Application Data
(60) Provisional application No. 63/351,921, filed on Jun. 14, 2022.

(51) Int. Cl. A61B 10/00

(52) U.S. Cl. CPC A61B 10/0051

(57) ABSTRACT

The invention provides a device for collecting a nasal secretion sample. The device includes a sample collection member comprising a material for collecting a predetermined volume of a nasal secretion sample. The device further includes a handle and an elongated support member extending between the handle and the sample collection member and for supporting the sample collection member. The device is adapted for a user holding the handle to locate the sample collection member in contact with tissue at a target location inside the nasal cavity where the sample collection member collects a predetermined volume of a nasal secretion sample.

### CROSS-REFERENCE TO RELATED APPLICATIONS TECHNICAL FIELD

This application is the National Stage of International Application No. PCT/AU2023/050525 filed Jun. 14, 2023, which claims the benefit of U.S. Application No. 63/351,921 filed Jun. 14, 2022, the entire disclosures each of which are hereby incorporated herein by reference.

### TECHNICAL FIELD

The present invention relates to a device and method for the collection of sinonasal secretion samples for subsequent analysis. In particular, the present invention relates to a device and method for the accurate volumetric sampling of nasal secretions. The invention is particularly adapted for use in relation to nasal secretion samples, however, it is to be appreciated that the invention may have broader application in relation to other samples such as sputum.

### BACKGROUND OF THE INVENTION

Sinonasal inflammatory conditions are characterised by the inflammation of mucous membranes that line the nasal cavity (rhinitis), the paranasal sinuses (sinusitis) or both (rhinosinusitis).

The most common causes of sinusitis and rhinosinusitis are viral infections, bacterial infections, and allergic immune responses such as hay-fever. In some rare cases, particularly in people with weakened immune systems, a fungal infection can be the cause. Rhinitis can be sub-categorised as allergic rhinitis and non-allergic rhinitis. Allergic rhinitis is triggered by allergens including dust, pollen and mould whereby non-allergic rhinitis may be caused by hormonal imbalances, environmental triggers such as perfume and fumes or consumption of medications including beta-blockers and ACE inhibitors.

Inflammation of the mucous membranes prevents mucous drainage, thereby causing mucous congestion. Symptoms associated with sinonasal inflammation include swelling, pressure, and sinus pain. Other symptoms commonly associated with sinusitis and rhinosinusitis include sneezing, fever, headaches, loss of smell, sore throat, mucus discharge and a cough.

These sinonasal inflammatory conditions are a common pathology experienced by humans, however, accurate diagnosis of the cause of these conditions remains a challenge. When a patient presents with symptoms associated with these conditions, a general practitioner (GP) would typically perform a physical examination by inspecting the face, ears, nose, throat, and neck to check for signs of swelling or congestion. If the symptoms are severe or persist, the GP would refer the patient to an Ear, Nose and Throat (ENT) specialist. ENT specialists often examine the sinonasal cavities using specialised tools such as endoscopes, speculums, and CT scanners. Following a physical examination confirming an inflammatory sinonasal condition, the cause of the condition is tested.

If an allergen is suspected to be the cause, the healthcare professional would perform an allergy test. If the cause is suspected as being viral or bacterial, then sampling techniques may be employed to collect samples from the sinonasal cavities for subsequent testing. There are two main categories of sinonasal secretion sampling techniques: bulk-surface fluid collection and focal-surface fluid collection.

Bulk-surface fluid collection includes forced exhalation of nasal secretions, nasal aspiration using a suction device or nasal lavage, whereby a saline solution is sprayed into the sinonasal cavities to collect the sample within the fluid.

Focal-surface fluid collection involves using absorptive materials such as cotton wool and swabbing techniques to recover sinonasal secretion samples. The most common method for obtaining sinonasal secretion samples includes an oropharyngeal and/or nasopharyngeal swab. The swab is inserted into the patient's nose and is aimed in a parallel direction to the nasal and septum floor. As long as there are no obstructions present within the nasal cavity, the swab will continue to move in this direction until it reaches the nasopharynx. Once the swab reaches the nasopharynx, the swab is either rotated to ‘scrape’ the mucous membranes or the sides of the nose are pinched to press the mucous membranes against the swab, therefore permitting sinonasal secretion absorption. Once the swab is removed from the patient's nose, it is stored in culture media or a buffer solution and transported in cold chain transport for subsequent testing.

Nasopharyngeal swabs are invasive and uncomfortable for the patient and trained medical personnel are often required to carry out the swab. These abrasive sampling techniques may further introduce contaminants into the sample such as blood. Alternatively, samples are taken directly from the sinonasal cavities during nasal endoscopy. The endoscopic observation of pus confirms the diagnosis of sinusitis and allows the specialist to place a small swab directly into the abnormal mucous. Endoscopic observation is also very invasive and uncomfortable and may require the patient to be sedated.

The testing of collected sinonasal samples is typically conducted in a laboratory and it can take some days for the healthcare professional and patient to receive the results. The laboratory testing requires low sample volumes (typically less than r equivalent to 50 ml). The processing typically involves diluting the sample with reagents and requires a specific final analyte concentration to ensure accuracy. Any deviations from the final analyte concentration can magnify the error margin, leading to inaccurate results and therefore insufficient diagnosis. As previously described, sampling devices often consist of an absorptive material to absorb the sinonasal secretions, however existing sampling devices are unable to collect standardised sample volumes consistent with the volumes required for accurate testing.

Furthermore, the existing sampling devices often require abrasive sampling techniques which can introduce contaminants such as blood into the sample and the size of the sampler and the depth of insertion may be incompatible with the patient, therefore leading to discomfort.

Accordingly, a need exists for a means for collecting a sinonasal secretion sample with volumetric control. A need also exists for a means for collecting a sinonasal secretion sample within a minimum and maximum sample amount to ensure assay accuracy. Furthermore, a need exists for a non-abrasive approach for collecting sinonasal biologic samples used to determine the cause of the identified inflammatory condition.

Any discussion of background art throughout the specification should in no way be considered as an admission that any of the documents or other material referred to was published, known or forms part of the common general knowledge.

Throughout the description and claims of this specification, the word “comprise” and variations of that word, such as “comprising” and “comprises” are not intended to exclude other additives, steps or integers.

### SUMMARY OF THE INVENTION

Accordingly, in one aspect, the present invention relates to a device for the collection of a nasal secretion sample. The device includes a handle and an elongated support member extending between the handle and a pair of frame members located towards a distal end of the elongated support member, wherein each of the frame members supports a sample collection member comprising a material for collecting a predetermined volume of a nasal secretion sample, and wherein the frame members are adapted to move relative to each other between open and closed positions, whereby the device is adapted for a user holding the handle to locate the sample collection member in contact with tissue at a target location inside the nasal cavity where the sample collection member collects a predetermined volume of a nasal secretion sample.

Preferably, the housing of the device encapsulates the sample collection member and deployment mechanism within the central cavity.

Various embodiments of the device are described and illustrated herein. In embodiments, the sample collection member comprises an absorptive material. Preferably, the sample collection member comprises multiple strips of an absorptive material with multiple snap points. In other embodiments, the sample collection member comprises a non-absorbent mesh strip with void volume.

Preferably, the geometry of the sample collection member allows for volumetric control during sample collection. Preferably, the sample collection member collects a nasal secretion sample within a minimum and maximum sample amount. In some embodiments, the sample collection member comprises a visual volume indicator means such as a dye indicator.

Preferably, the sample collection member comprises an absorptive material adapted for absorbing the nasal secretion sample. Preferably, the sample collection member comprises a length of an absorptive material with one or more points for detaching a part of the absorptive material for adjusting the predetermined absorption volume of the sample collection member. In embodiments, the sample collection member comprises a non-absorbent mesh strip comprising a void volume for retaining the nasal secretion sample.

Preferably, the sample collection member is configured for collecting a nasal secretion sample within a minimum and maximum volume range. In embodiments, the sample collection member comprises a visual indicator that is responsive to collection of a predetermined volume of the nasal secretion by the sample collection member to provide a visual indication. In embodiments, the visual volume indicator includes a dye indicator.

Preferably, the deployment mechanism includes a manually operable handle connected to the sample collection member, wherein the handle is movable relative to the housing to move the sample collection member between retracted and deployed positions.

Preferably, the housing includes an excess sample removal device for removing excess sample on a surface of the sample collection member. In embodiments, the excess sample removing device includes a wiper for contacting the surface of the sample collection member as the sample collection member moves from the deployed position to the retracted position for wiping excess nasal secretion sample from the surface. Preferably, the wiper includes a wall surrounding an aperture having a cross-section that is dimensioned to be equivalent to a cross section of the sample collection member containing no more than the predetermined volume of sample to thereby wipe excess sample from the surface of the sample collection member as the sample collection member is drawn through the aperture.

Preferably, in the deployed position the sample collection member protrudes from the housing for contact with a nasal secretion sample collected in a container or in absorbent sample collection material.

In embodiments, the housing is configured to be inserted into a container comprising a desiccant to dry the sample and maintain low relative humidity. Preferably, the housing is configured to be inserted into a container comprising a preservative such as a buffer solution.

In embodiments, the elongated support member includes an aperture at a distal end thereof for containing the sample collection member therewithin, wherein at least one side of the sample collection member is exposed for contacting with nasal cavity tissue.

Preferably, the elongated support member includes a pair of arms each supporting a respective sample collection member at a distal end thereof, wherein the handle includes a pair of grip members at the proximal ends of the arms, wherein manually closing the grip members together closes the sample collection members together for insertion into one nostril or two nostrils simultaneously.

Preferably, the sample collection members are biased apart for contacting with nasal tissue upon releasing the grip members when the sample collection members are located in the nasal cavity.

In embodiments, the device further includes one or more pivoting frame members located towards a distal end of the elongated support member, wherein each pivoting frame member supports at least a portion of one or more of the sample collection members.

Preferably, the pivoting frame members have an arcuate shape and are pivotally connected to the elongated support member to be movable relative to each other between an open position and a closed position.

Preferably, the pivoting frame members are biased towards the open position and wherein upon insertion into the nasal cavity the frame members and the one or more sample collection members supported thereon come into contact with the nasal tissue and are thereby urged towards the closed position.

In embodiments, the device includes one or more expanding frame members located towards a distal end of the elongated support member, wherein each expanding frame member supports at least a portion of one or more of the sample collection members.

Preferably, the expanding frame members have a resting state in which the frame members are closed together and an expanded state in which the frame members are opened apart from each other.

Preferably, in the resting state the frame members are flat and in the expanded state the frame members are arcuate or vice versa.

In embodiments, the device includes a mechanism for selectively expanding or closing the frame members, the mechanism including a pair of manually operable handle members at the proximal end of the elongated support member, wherein manually closing the handle members together opens the frame members and manually opening the handle members closes the frame members or vice versa.

Preferably, the frame members are biased towards the resting state and wherein upon insertion into the nasal cavity the frame members are movable to the expanded state wherein the frame members and the one or more sample collection members supported thereon come into contact with the nasal tissue.

Embodiments of the device can be used for collecting a precise volume of a nasal secretion sample previously collected from the patient in a container or another suitable vessel to ensure assay accuracy.

In another aspect, the invention relates to a method for the collection of a nasal secretion sample. The method includes: locating a sample collection device in a nasal cavity of a patient, the device including an elongated support member extending between a handle and a pair of frame members located towards a distal end of the elongated support member, wherein the frame members are adapted to move relative to each other between open and closed positions and wherein each of the frame members supports a sample collection member comprising a material for collecting a predetermined volume of a nasal secretion sample; contacting the sample collection members with tissue at a target location inside the nasal cavity; collecting a predetermined volume of a nasal secretion sample in the sample collection member; and removing the device from the nasal cavity of the patient.

Preferably, the method includes wiping excess nasal secretion sample from the sample collection member during retraction of the sample collection member.

Preferably, the method includes contacting the deployed sample collection member contact with a nasal secretion sample collected in a container or in absorbent sample collection material.

### BRIEF DESCRIPTION OF THE FIGURES

The present invention will now be described in more detail with reference to preferred embodiments illustrated in the accompanying figures, wherein:

FIG. 1 illustrates a perspective view of a sample collection device in accordance with an embodiment of the invention including a housing, deployment mechanism, sample collection member and wiping mechanism.

FIG. 2 illustrates a front view of the sample collection device of FIG. 1.

FIGS. 3 and 4 illustrate a perspective and front view of the sample collection device of FIGS. 1 with the sample collection member deployed.

FIGS. 5 and 6 illustrate and perspective and front view of the sample collection device of FIG. 1, illustrating the sample collection member in its retracted states.

FIG. 7 illustrates a perspective view of the sample collection device in accordance with another embodiment of the invention, wherein the sample collection member is retracted.

FIG. 8 illustrates a perspective view of the sample collection device of FIG. 7, wherein the sample collection member is deployed.

FIG. 9 illustrates a perspective view of the sample collection device in accordance with another embodiment of the invention, wherein the deployment mechanism is a scroll wheel.

FIG. 10 illustrates a partially exploded perspective view of the sample collection device of FIG. 9, wherein the elongate member is deployed.

FIG. 11 illustrates a perspective view of the sample collection device in accordance with yet another embodiment of the invention, wherein the deployment mechanism comprises a slide.

FIG. 12 illustrates a perspective view of the sample collection device of FIG. 11, wherein the sample collection member is deployed.

FIG. 13 illustrates a perspective view of a sample collection device in accordance with another embodiment of the invention including an elongated support member including an aperture at a distal end thereof for containing the sample collection member.

FIG. 14 illustrates a perspective view of a sample collection device in accordance with yet another embodiment of the invention wherein the elongated support member includes a pair of arms each supporting a respective sample collection member at a distal end thereof.

FIGS. 15 and 16 illustrate perspective views of a sample collection device in accordance with yet another embodiment of the invention including a pair of pivoting frame members located towards a distal end of an elongated support member, wherein each pivoting frame member supports at least a portion of a sample collection member.

FIGS. 17 and 18 illustrate perspective views of a sample collection device in accordance with yet another embodiment of the invention including wherein parts of a sample collection member are supported by a pair of expanding frame members located towards a distal end of an elongated support member, wherein each expanding frame member supports at least a portion of a sample collection member, wherein FIG. 17 illustrates the expanding frame members in a resting state and FIG. 18 illustrates the expanding frame members in an expanded state.

FIGS. 19 and 20 illustrate section views of the device of FIGS. 17 and 18, wherein FIG. 19 illustrates the expanding frame members in a resting state and FIG. 20 illustrates the expanding frame members in an expanded state.

FIG. 21 illustrates a perspective view of a sheath in use with the device of FIGS. 17 to 20.

### DETAILED DESCRIPTION

The present invention relates to a device and method for the collection of nasal secretion samples. Several embodiments are described herein, however the sample collection device broadly comprises a device for collecting a nasal secretion sample, the device including a sample collection member comprising a material configured for collecting a predetermined volume of a nasal secretion sample, a handle, and an elongated support member extending between the handle and the sample collection member and for supporting the sample collection member, wherein the device is adapted for a user holding the handle to locate the sample collection member in contact with tissue at a target location inside the nasal cavity where the sample collection member collects a predetermined volume of a nasal secretion sample.

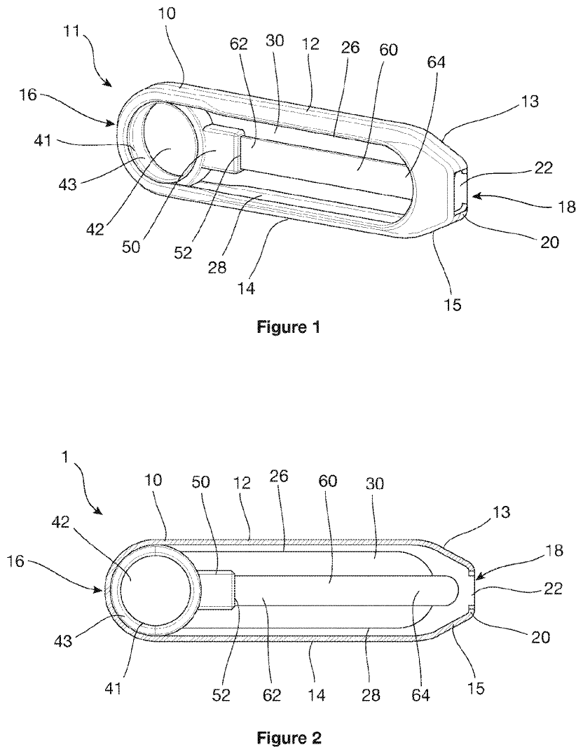

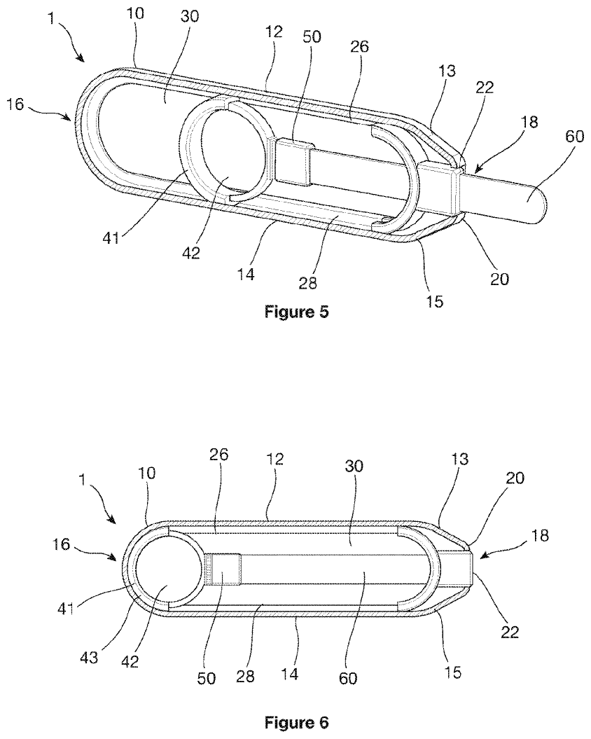

FIGS. 1-6 illustrate diagrammatic representations of a device 1 for the collection of a nasal secretion sample in accordance with an embodiment of the invention. The device 1 comprises a housing 10 with elongated wall members 12, 14 extending between a proximal end 16 and a distal end 18. The wall members 12, 14 are arranged parallel and spaced apart and define a central cavity 30. The central cavity 30 is elongated and extends from the proximal end 16 to the distal end 18 of the housing 10. The housing 10 is oblong in shape and the wall members 12, 14 are generally linear and extend from the proximal end 18 which is curved or arcuate and lead to tapering end portions 13, 15. The end portions 13, 15 taper towards an end wall 20 at the distal end 18. The end wall 20 of the housing 10 includes an aperture 22.

The device 1 includes a deployment mechanism 40 including a manually operable handle 41. The handle 41 includes an annular wall 43 defining a ring-shaped opening 42. The handle 41 is slidably mounted within the central cavity 30 between the elongated wall members 12, 14. The elongated wall members 12, 14 include respective linear tracks 26, 28 for guiding the movement of the handle 41 back and forth in a linear direction between the proximal and distal ends 16, 18 of the housing 10. Edges of the handle 41 engage the linear tracks 26, 28 of the wall members 12, 14.

The handle 41 is configured to receive one or more fingers of a user within the ring-shaped opening 42, allowing the user to slide the handle 41 within the central cavity 30 between the proximal end 16 and the distal end 18 by a pulling/pushing motion. The handle 41 is configured to remain in a fixed position at the proximal end 16, such as by the operation of a detent, until force is manually applied by the user to slide the handle 41 to the distal end 18.

The device 1 includes a sample collection member 60 for the collection of a nasal secretion sample. The sample collection member 60 comprises a body 61 extending longitudinally between a proximal end 62 and a distal end 64. The sample collection member 60 is configured for absorbing a predetermined volume of a nasal secretion sample. Preferably, the sample collection member 60 absorbs a sample volume within 10-200 ml or a predetermined volume suitable for subsequent assay accuracy. Preferably, the material of the sample collection member 60 is absorptive and paper based. In other embodiments, the material of the sample collection member 60 is nylon based, cotton based, polyurethane based, porous polymer based, foam based, cellulosed based, hydrophilic, or hydrophobic porous material based, open or closed cell materials based, flexible polymeric materials based or consists of another material suitable for volumetric control. Furthermore, the sample collection member 60 preferably comprises multiple strip zones with the same or varying volume capacities that are divided by snap points. In another embodiment, the sample collection member 60 comprise a non-absorbent mesh strip with void volume.

Accordingly, the housing 10 contains a deployment mechanism 40 operable to move the sample collection member 60 between retracted and deployed positions, wherein in the retracted position the housing 10 encapsulates the sample collection member 60 and in the deployed position the sample collection member 60 protrudes from the housing 10 for insertion into a nasal cavity for collecting a nasal secretion sample.

The proximal end 62 of sample collection member 60 is connected to the handle 41. In the embodiment of FIGS. 1 to 3, the handle 41 includes a support member 50 for connecting the sample collection member 60 to the handle 41. The support member 50 is affixed to the annular wall 43 surrounding the ring-shaped opening 42. The support member 50 includes a longitudinal recess 52 for receiving and securely retaining the proximal end 62 of the sample collection member 60 therewithin. The support member 50 is preferably integrally formed with the annular wall 43 of the handle 41. In use, the support member 50 is dimensioned to be receivable within the aperture 22 within the end wall 20 of the housing 10.

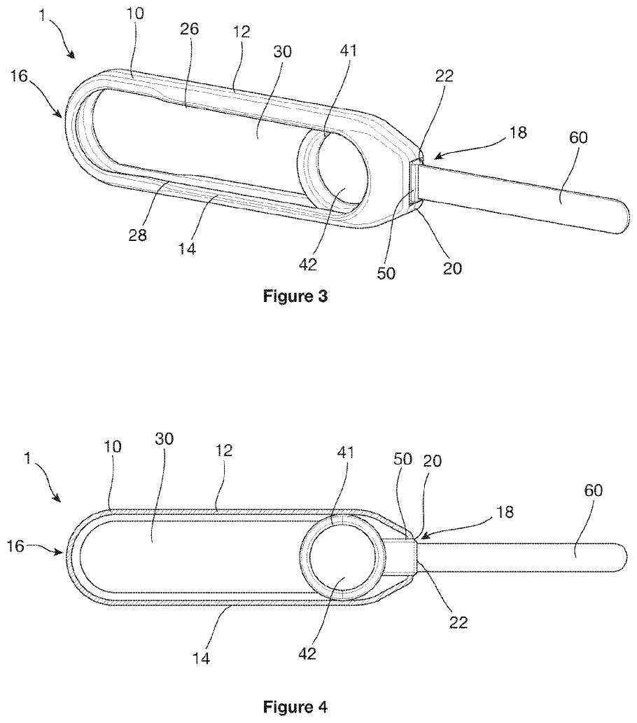

As illustrated in FIGS. 1 and 2, the sample collection member 60 is located fully within the central cavity 30 of the housing 10, which herein is referred to as the ‘retracted position’. In the retracted position, the handle 41 is at the proximal end 16 of the housing 10. In use, the handle 41 is moved from the proximal end 16 to the distal end 18 of the housing 10. Movement of the handle 41 to the distal end 18 of the housing 10 moves the sample collection member 60 from the central cavity 30, through the aperture 22 in the end wall 20 and substantially out of the housing 10. As illustrated in FIGS. 3 and 4, when handle 41 reaches the distal end 18 and cannot move any further, the sample collection member 60 is located external to end wall 20 of the housing, which herein is referred to as the ‘deployed position’. When the sample collection member 60 is in the deployed position, the support member 50 is located within the aperture 22 within the end wall 20 of the housing 10. In embodiments, the device 1 can include a mechanism to releasably hold the handle 41 and the sample collection member 60 in the deployed position.

When in the deployed position, the sample collection member 60 is adapted to collect a respiratory secretion sample. Preferably, the sample collection member 60 is deployed directly into the nasal cavity of a patient to minimise sample contamination. Preferably, the dimensions of the sample collection member 60 are determined so as to fit smaller nostrils comfortably and allow for sampling of different age groups. The speed with which the handle 41 is moved to deploy the sample collection member 60 can be controlled by the user to reduce patient discomfort that may result from sudden insertion of the sample collection member 60, particularly if the patient has a deviated septum or polyps.

The sample collection member 60 preferably comprises a visual volume indicator means such as a dye indicator. The visual volume indicator is responsive to when a predetermined volume of the nasal secretion has been collected by the sample collection member 60 to provide a visual indication during or subsequent to sample collection. The visual volume indicator is selected from the group including polymeric beads, peroxide or oxidation reactions, leuco dyes, food dyes and change of refractive index of the material.

After the sample is collected by the sample collection member 60, the user can manually slide the handle 41 from the distal end 18 to the proximal end 16 of the housing 10. In so doing, the sample collection member 60 is drawn back through the aperture 22 within the end wall 20 of the housing 10 until the sample collection member 60 is located fully within the central cavity 30 of the housing 10, which herein is referred to as the ‘retracted position’.

The aperture 22 is surrounded by the end wall 20 and has a cross-section that is dimensioned to be equivalent to a cross section of the sample collection member 60 when the sample collection member 60 contains no more than the predetermined volume of sample. Because the sample collection member 60 may swell upon absorption of the sample, the cross-sectional dimensions of the aperture 22 may be slightly larger than the cross-sectional dimensions of the unused sample collection member 60. However, a relatively tight tolerance is provided between the cross-sectional dimensions of the sample collection member 60 and the cross-sectional dimensions of the aperture 22. The end wall 20 surrounding the aperture 22 is operable to wipe excess sample from an external surface of the sample collection member 60 as the sample collection member 60 is drawn through the aperture 22. Removing excess nasal secretion sample from the sample collection member 60 is advantageous for maintaining volumetric control and consequently, assay accuracy.

To reduce instability and degradation of the collected sample, particularly when stored at room temperature, the housing 10 of device 1 preferably contains a desiccant means to dry the sample and maintain low relative humidity. Alternatively, the housing 10 is adapted to be placed into a container that contains a desiccant means to dry the sample and maintain low relative humidity. The desiccant means is preferably silica gel, calcium oxide, calcium sulphate, montmorillonite or another suitable means. Alternatively, the collected sample is preserved in a buffer solution or refrigerated for same day use or is lyophilised or frozen for long-term use.

The foregoing further embodiments of the invention comprise like or equivalent features as described in the embodiment of FIGS. 1 to 6 described above. Like features are described in like terms. For the sake of conciseness, only features of the foregoing further embodiments of the invention that differ substantially in form and/or function from the embodiment of FIGS. 1 to 6 are described in detail below.

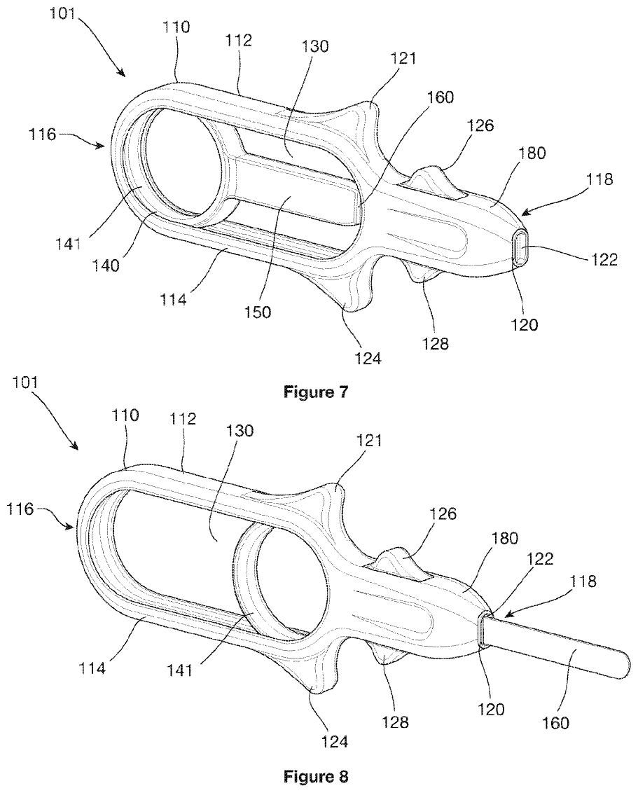

In FIGS. 7 and 8, the device 101 comprises a housing 110 with a proximal end 116 and a distal end 118 and wall members 112, 114 that are parallel and spaced apart and define a central cavity 130. In this embodiment, the wall members 112, 114 comprise a set of outwardly extending wing portions 121, 124 located towards the distal end 118. The distal end 118 of the housing 110 comprises an elongated nose portion 180 with an additional set of winglets 126, 128. At least part of the nose portion 180 can be inserted in the nose or throat of a patient.

The central cavity 130 houses the deployment mechanism 140 including the handle 141, support member 150 and sample collection member 160. The end wall 120 comprising the aperture 122 for wiping excess sample from the sample collection member 160 is located at a distal tip of the elongated nose portion 180. As illustrated in FIG. 7, the sample collection member 160 is in the retracted position in which the sample collection member 160 is located fully within the central cavity 130 of the housing 110. In use, the handle 141 is moved towards the distal end 118 of housing 110, deploying the sample collection member 160 from the elongated nose 180, through the aperture 122 and to the deployed position. Once the nasal secretion sample is collected, the sample collection member 160 is withdrawn back through the aperture 122 to the retracted position whereby excess nasal secretion is wiped from the sample collection member 160 by the end wall 120 surrounding the aperture 122.

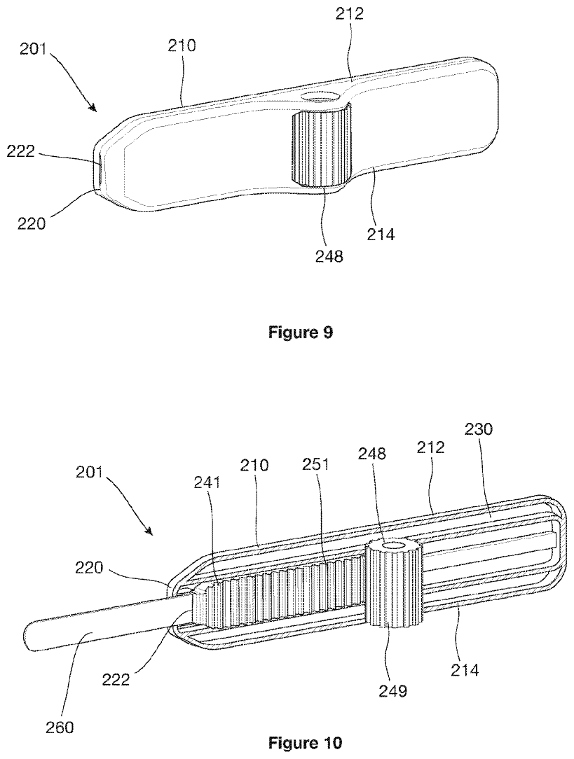

The embodiments as illustrated in FIGS. 9 and 10 and FIGS. 11 and 12 vary in the configuration of the deployment mechanism. Referring to the embodiment of the device 201 illustrated in FIGS. 9 and 10, the deployment mechanism 240 includes a scroll wheel 248. The wall members 212, 214 of the housing 210 are configured to come together to completely enclose the central cavity 230. FIG. 10 illustrates the device 201 with one of the wall members 214 removed to reveal the internal cavity 230 and the support handle 250. In use, the sample collection member 260 is deployed by manually rotating the scroll wheel 248. The support member 250 handle has a set of linear teeth 251 that are engaged by a set of teeth 249 provided about the circumference of the scroll wheel 248. When the scroll wheel 248 is rotated in the opposite direction, the sample collection member 260 retracts back within the housing 210 whereby excess nasal secretion is wiped from the sample collection member 260 by the end wall 220 surrounding the aperture 222.

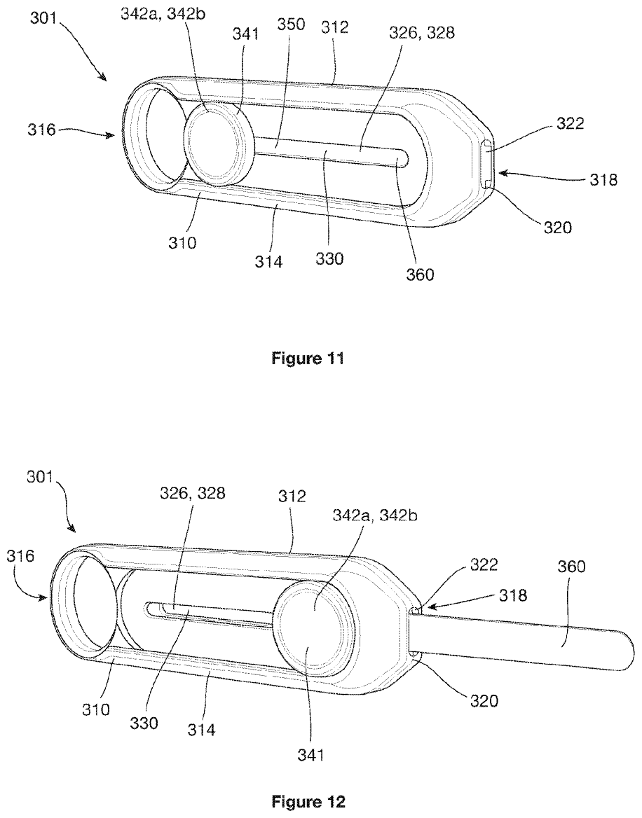

Referring to the embodiment of the device 301 illustrated in FIGS. 11 and 12, the handle 341 of the deployment mechanism 340 includes a pair of opposite solid gripping surfaces 342a, 342b configured to be gripped between the thumb and forefinger of a user. The wall members 312, 314 of the housing 310 are configured to come together to substantially completely enclose the central cavity 330. The user grips the gripping surfaces 342a, 342b to slide the handle 341 along elongated, linear openings 326, 328 through the wall members 312, 314. The gripping surfaces 342a, 342b of the handle 341 are coupled through the linear openings 326, 328 to the support member 350 which is located within the cavity 330. The support member 350 is coupled to the sample collection member 360 in the manner described above. The sample collection member 360 is deployed when the user grips the gripping surfaces 342a, 342b to slide the handle 341 to the distal end 318 of the housing 310. The sample collection member 360 is retracted by sliding the sliding the handle 341 from the distal end 318 to the proximal end 316 of the housing 310. The retracting sample collection member 360 is withdrawn through the aperture 322 whereby excess nasal secretion is wiped from the sample collection member 360 by the end wall 320 surrounding the aperture 322.

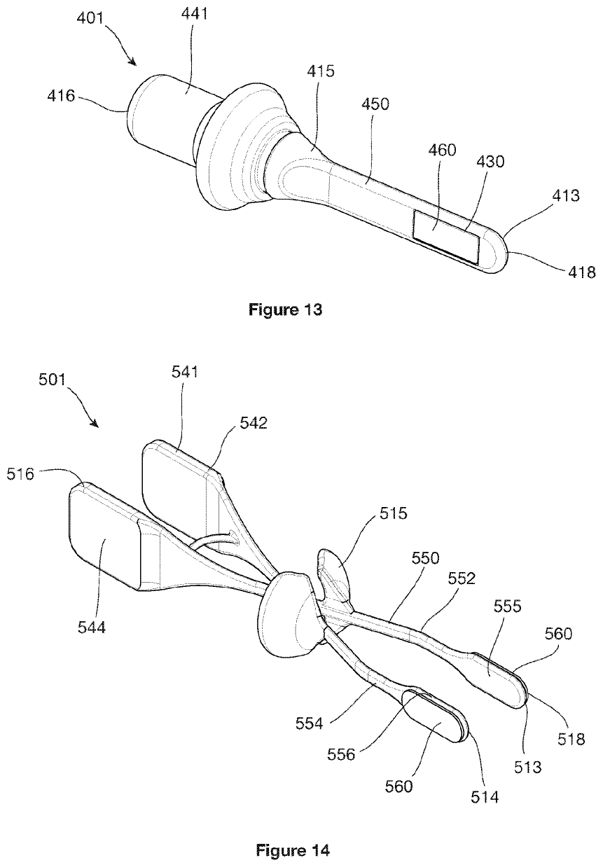

FIG. 13 Illustrates an embodiment of the device 401 including a sample collection member 460, a handle 441 and an elongated support member 450 extending between the handle 441 and the sample collection member 460. The elongated support member includes an aperture (430) at a distal end 418 thereof for containing the sample collection member therewithin, wherein at least one side of the sample collection member is exposed for contacting with nasal cavity tissue.

The elongated support member 450 extends between the handle 441 at a proximal end 416 and extends to a distal end 418 including a distal tapering portion 413 for insertion into the nasal cavity. The elongated member 450 further includes a proximal tapering portion 415 immediately adjacent to the handle 441 to limit the extent to which the device 401 can be inserted into the nasal cavity.

In use, a user holds the handle 441 and inserts the distal end 418 into the nasal cavity which brings the sample collection member 460 into contact with tissue at a target location inside the nasal cavity where the sample collection member 460 collects a predetermined volume of a nasal secretion sample.

FIG. 14 Illustrates an embodiment of the device 501 including a sample collection member 560 wherein the elongated support member 550 includes a pair of arms 552, 554 each supporting a respective sample collection member 560 at a distal end thereof. The handle 541 includes a pair of grip members 542, 544 at the proximal ends of the arms 552, 554 and a pair of frame members 555, 556 at distal ends of the arms 552, 554 for supporting the sample collection members 560. The device 501 is operable so that manually closing the grip members 542, 544 together closes the sample collection members 560 together for insertion into the nasal cavity. The sample collection members 560 are biased apart for contacting with nasal tissue upon releasing the grip members 542, 544 when the sample collection members are located in the nasal cavity.

The frame members 555, 556 at distal ends of the arms 552, 554 include distal tapering ends 513, 514 for insertion into the nasal cavity. The elongated member 550 further includes a proximal tapering portion 515 immediately adjacent to the handle 541 to limit the extent to which the device 501 can be inserted into the nasal cavity.

In use, a user holds the grip members 542, 544 of the handle 541 and inserts the distal end 518 into the nasal cavity, either by insertion of both of the arms 552, 554 into a single nostril or into two nostrils simultaneously, which brings the sample collection members 560 into contact with tissue at a target location inside the nasal cavity where the sample collection member 560 collects a predetermined volume of a nasal secretion sample.

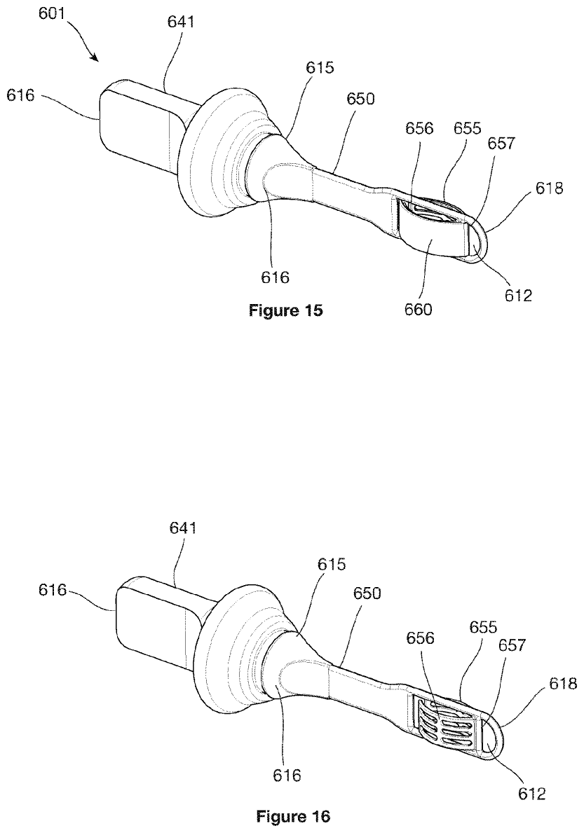

FIGS. 15 to 16 illustrate an embodiment of the device 601 including a sample collection member 660, a handle 641 and an elongated support member 650 extending between the handle 641 and the sample collection member 660. A pair of pivoting frame members 655, 656 are located towards the distal end 618 of the elongated support member 650. Each pivoting frame member 655, 656 supports at least a portion of the one or more of the sample collection members 660.

In the embodiment illustrated in FIGS. 15 and 16, an elongated sample collection member 660 passes through an aperture 612 at the distal end 618 of the elongated support member 650. Parts of the sample collection member 660 are supported by the pair of pivoting frame members 655, 656. Ends of the sample collection member 660 are affixed to the elongate support member 650 adjacent to the pivoting frame members 655, 656 or are otherwise fixed to the pivoting frame members 655, 656 themselves such as with an adhesive or mechanical attachment.

The pivoting frame members 655, 656 have an arcuate shape and are connected to the elongated support member 650 at a pivotal connection 657. The pivoting frame members 655, 656 are therefore movable relative to each other between an open position and a closed position. In the embodiment illustrated in the figures, the pivoting movement of the frame members 655, 656 is achieved from the resiliently flexible property of the material forming the frame members 655, 656. However, in other embodiments, the pivoting movement is achieved with a hinge mechanism and a biasing means.

The frame members 655, 656 are biased towards the open position and wherein upon insertion into the nasal cavity the frame members 655, 656 and the one or more sample collection members 660 supported thereon come into contact with the nasal tissue and are thereby urged towards the closed position.

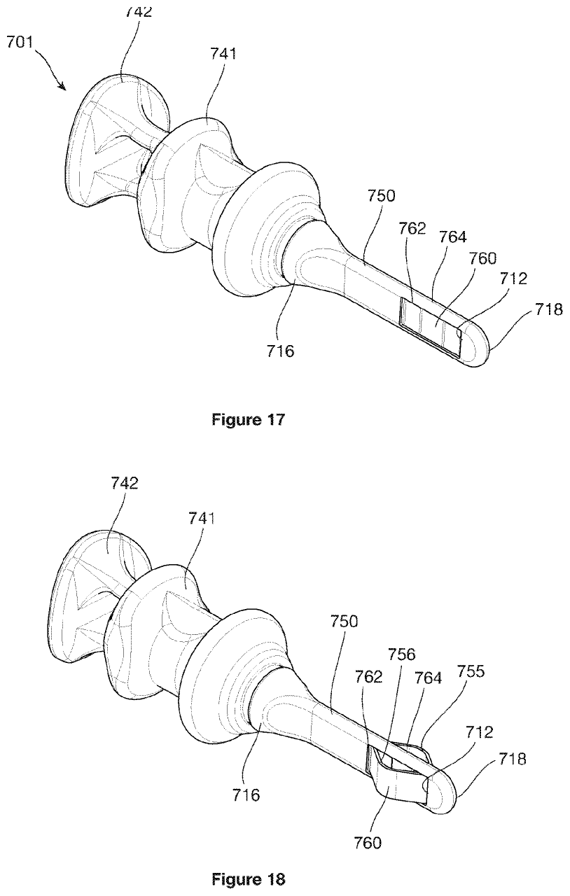

FIGS. 17 to 20 illustrate an embodiment of the device 701 including a sample collection member 760, a handle 741 and an elongated support member 750 extending between the handle 741 and the sample collection member 760. One or more expanding frame members 755, 756 are located towards a distal end of the elongated support member 750. Each expanding frame member 755, 756 supports at least a portion of one or more of the sample collection members 760.

The frame members 755, 756 are located in one or more channels or recesses 762 within the elongated support member 750 towards the distal end 718 thereof. The frame members 755, 756 are located in channels or recesses 762, 764 on opposite sides of the elongated support member 750 towards the distal end 718 thereof and face away from each other.

In the embodiment of FIGS. 17 to 20, the elongated sample collection member 760 passes through an aperture 712 at the distal end 718 of the elongated support member 750. Parts of the sample collection member 760 are supported by the pair of frame members 755, 756. Ends of the sample collection member 760 are affixed to the elongate support member 750 adjacent to a proximal end of the frame members 755, 756 or are otherwise fixed to the frame members 755, 756 themselves such as with an adhesive or mechanical attachment.

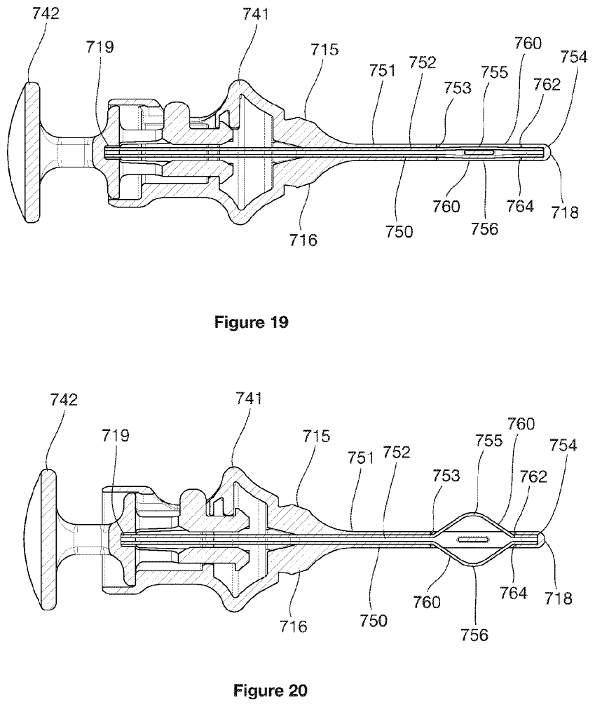

In an embodiment, the expanding frame members 755, 756 have a resting state in which the frame members 755, 756 are closed together, as illustrated in FIGS. 17 and 19, and an expanded state in which the frame members 755, 756 are opened apart from each other, as illustrated in FIGS. 18 and 20. Accordingly, in the resting state as illustrated in FIGS. 17 and 19 the frame members 755, 756 are flat. In the expanded state as illustrated in FIGS. 18 and 20 the frame members 755, 756 are arcuate.

The device 701 includes a mechanism for selectively expanding or closing the frame members 755, 756. The mechanism includes a pair of manually operable handle members 741, 742 at the proximal end of the elongated support member 750. The handle members 741, 742 are configured to be manually closable together to thereby open the frame members 755, 756, as illustrated in FIGS. 18 and 20, and to be manually openable apart to thereby close the frame members 755, 756 together, as illustrated in FIGS. 17 and 19.

As illustrated in FIGS. 19 and 20, the elongated support member 750 includes an outer tube member 751 and an inner tube member 752. The outer tube member 751 is connected at a proximal end 716 to a distal one of the handle members 741 and the inner tube member 752 is connected at a proximal end 719 to a proximal one of the handle members 742. The inner tube member 752 is slidable within the outer tube member 751 whereby movement of the handle members 741, 742 axially relative to each other causes the inner tube member 752 to slide axially relative to the outer tube member 751.

The frame members 755, 756 comprise, are integrally formed with or are connected to a distal end 753 of the inner tube member 752. The frame members 755, 756 are comprised of a flexible material, that is preferably also resilient, and are located between the distal end 753 of the inner tube member 752 and a distal end 754 of the outer tube member 751 at the distal end 718 of the elongated support member 750.

In use, when the handle members 741, 742 are manually closed together, the inner tube member 752 slides inside the outer tube member 751 to thereby compress the frame members 755, 756 between an end of the inner tube member 752 and the distal end of the elongated support member 750. The frame members 755, 756 are thereby forced into an expanded state in which the frame members 755, 756 are opened apart from each other as illustrated in FIGS. 18 and 20.

Conversely, movement of the handle members 741, 742 apart from each other causes the inner tube member 752 to slide in the opposite direction inside the outer tube member 751 to thereby remove any compression force from the frame members 755, 756. Either due to a resilient property of the frame members 755, 756 or due to a force applied through the inner tube member 751 or both, the frame members 755, 756 are closed together, as illustrated in FIGS. 17 and 19.

In another embodiment, the expanding frame members 755, 756 have a resting state in which the frame members 755, 756 are in the expanded state as illustrated in FIGS. 18 and 20. Accordingly, either due to a force applied by the inner tube member 751 or an external force applied directly to the frame members 755, 756, such as upon insertion into a nasal cavity, the frame members 755, 756 are closed together, as illustrated in FIGS. 17 and 19.

Movement of the handle members 741, 742 apart from each other causes the inner tube member 752 to slide inside the outer tube member 751 to apply a tensile force to force the frame members 755, 756 into the closed position as illustrated in FIGS. 17 and 19. Upon releasing the handle members 741, 742 the expanding frame members 755, 756 assume the expanded state as illustrated in FIGS. 18 and 20. In another embodiment, the device 701 is configured so that movement of the handle members 741, 742 together forces the frame members 755, 756 into the closed position as illustrated in FIGS. 17 and 19.

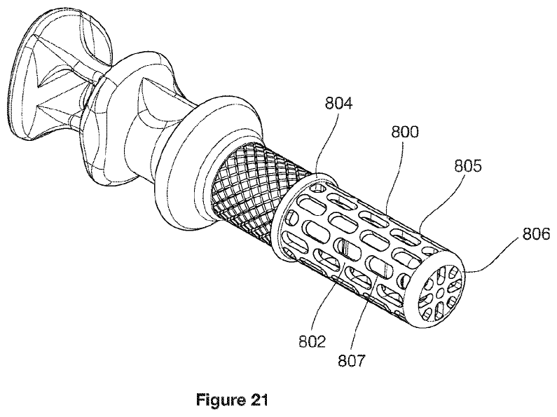

As illustrated in the embodiment of FIG. 21, a sheath 800 is adapted for use with the device 701 of FIGS. 17 to 20. In particular, the sheath 800 comprises a cylindrical tube member 805 that is closed at a distal end 806 and is open at a proximal end 804. The tube member 805 is adapted to receive the elongated support member 750 of the device 701 through the opening at the proximal end 804. The tube member 805 includes a cylindrically shaped wall 802 including a plurality of apertures 807 permitting airflow through the wall 802. The airflow through the wall 802 of the tube member 805 permits air drying of the sample contained within the collection member 760 while also protecting the sample.

Embodiments of the sheath 800 of FIG. 21 can also be configured for use with the devices 401, 601 and 701 of FIGS. 13 and 15 to 20.

In use, the embodiment of the device 1, 101, 201, 301, 401, 501 and 701 can be used to collect a predetermined volume of a nasal secretion sample previously collected from the patient in a container or another suitable vessel. The user can deploy the sample collection member 60, 160, 260, 360 into nasal secretions pooled into a container or tube or absorbed into an absorbent material or swab. Accordingly, the device 1, 101, 201, 301 can be employed to collect a sample of precise volume from an amount of sample collected form a patient into a container or in some other absorbent material or swab so as to ensure assay accuracy.

Although the invention has been described with reference to specific examples, it will be appreciated by those skilled in the art that the invention may be embodied in many other forms, in keeping with the broad principles and the spirit of the invention disclosed herein.

1. A device for collecting a nasal secretion sample, the device including:
a handle; and
an elongated support member extending between the handle and a pair of frame members located towards a distal end of the elongated support member, wherein each of the frame members supports a sample collection member comprising a material for collecting a predetermined volume of a nasal secretion sample; and
wherein the frame members are adapted to move relative to each other between open and closed positions, whereby the device is adapted for a user holding the handle to locate the sample collection member in contact with tissue at a target location inside the nasal cavity where the sample collection member collects a predetermined volume of a nasal secretion sample.

2. The device of claim 1, wherein the sample collection member comprises an absorptive material adapted for absorbing the nasal secretion sample.

3. (canceled)

4. (canceled)

5. The device of claim 1, wherein the sample collection member is configured for collecting a nasal secretion sample within a minimum and maximum volume range.

6. The device of claim 1, wherein the sample collection member comprises a visual indicator that is responsive to collection of a predetermined volume of the nasal secretion by the sample collection member to provide a visual indication.

7. The device of claim 6, wherein the visual volume indicator includes a dye indicator.

8. (canceled)

9. (canceled)

10. (canceled)

11. (canceled)

12. (canceled)

13. (canceled)

14. The device of claim 1, wherein the device is configured to be inserted into a container comprising a desiccant to dry the sample and maintain low relative humidity.

15. The device of claim 1, wherein the device is configured to be inserted into a container comprising a preservative such as a buffer solution.

16. (canceled)

17. The device of claim 1, wherein the elongated support member includes a pair of arms at a distal end thereof supporting the pair of frame members.

18. The device of claim 17, wherein the pair of frame members are biased apart for contacting the nasal tissue with the sample collection members upon releasing the grip members when the sample collection members are located in the nasal cavity.

19. (canceled)

20. The device of claim 1, wherein the frame members have an arcuate shape and are pivotally connected to the elongated support member to be movable relative to each other between an open position and a closed position.

21. The device of claim 20, wherein the frame members are biased towards the open position and wherein upon insertion into the nasal cavity the frame members and the one or more sample collection members supported thereon come into contact with the nasal tissue and are thereby urged towards the closed position.

22. (canceled)

23. The device of claim 1, wherein the frame members have a resting state in which the frame members are closed together and an expanded state in which the frame members are opened apart from each other.

24. The device of claim 23, wherein in the resting state the frame members are flat and in the expanded state the frame members are arcuate or vice versa.

25. The device of claim 23, including a mechanism for selectively expanding or closing the frame members, the mechanism including a pair of manually operable handle members at the proximal end of the elongated support member, wherein manually closing the handle members together opens the frame members and manually opening the handle members closes the frame members or vice versa.

26. The device of claim 23, wherein the frame members are biased towards the resting state and wherein upon insertion into the nasal cavity the frame members are movable to the expanded state wherein the frame members and the one or more sample collection members supported thereon come into contact with the nasal tissue.

27. A method for the collection of a nasal secretion sample, the method including:
locating a sample collection device in a nasal cavity of a patient, the device including an elongated support member extending between a handle and a pair of frame members located towards a distal end of the elongated support member, wherein the frame members are adapted to move relative to each other between open and closed positions and wherein each of the frame members supports a sample collection member comprising a material for collecting a predetermined volume of a nasal secretion sample;
contacting the sample collection members with tissue at a target location inside the nasal cavity;
collecting a predetermined volume of a nasal secretion sample in the sample collection member; and
removing the device from the nasal cavity of the patient.

28. (canceled)

29. (canceled)

30. The device of claim 17, wherein the handle includes a pair of grip members at the proximal ends of the arms, wherein manually closing the grip members together closes the sample collection members together for insertion into one nostril or two nostrils simultaneously.
